(19) United States
(12) Patent Application Publication
(10) Pub. No.: US 2022/0056419 A1
(43) Pub. Date: Feb. 24, 2022

(54) IN VITRO MODEL OF LIVER STEATOSIS AND FIBROSING NON-ALCOHOLIC STEATOHEPATITIS

(71) Applicant: GENFIT, LOOS (FR)

(72) Inventors: GUILLAUME VIDAL, SAINT-ANDRE-LEZ-LILLE (FR); FLORIANE LIMOGE, MARCQ-EN-BAROEUL (FR); JULIETTE DROUARD, WAHAGNIES (FR); ROBERT WALCZAK, LILLE (FR); CAROLE BELANGER, MONT-ROYAL (CA)

(21) Appl. No.: 17/414,966

(22) PCT Filed: Dec. 18, 2019
(86) PCT No.: PCT/EP2019/086136

§ 371 Date: Jun. 17, 2021

(30) Foreign Application Priority Data
Dec. 21, 2018 (EP) 18306837.8

(51) Int. Cl. C12N 5/071; G01N 33/50

(52) U.S. Cl. CPC C12N 5/0671; G01N 33/5088; G01N 33/5038; G01N 2500/10; C12N 2501/135; C12N 2501/33; C12N 2500/36; C12N 2501/25; C12N 2500/34

(57) ABSTRACT

The present invention relates to compositions and methods for preparing in vitro models of non-alcoholic fatty liver disease, and more particularly of liver steatosis and fibrosing non-alcoholic steatohepatitis (NASH).

The present invention relates to compositions and methods for preparing in vitro models of non-alcoholic fatty liver disease, and more particularly of liver steatosis and fibrosing non-alcoholic steatohepatitis (NASH).

Non-alcoholic fatty liver disease (NAFLD) is a rapidly emerging public health crisis, affecting up to ⅓ of the U.S. population, 75% of type 2 diabetics, and 95% of obese individuals. Early in the NAFLD disease spectrum, non-alcoholic fatty liver (NAFL) is benign and asymptomatic. NAFL is characterized by accumulation of fat within the liver's hepatocytes. However, this benign phenotype can progress to non-alcoholic steatohepatitis (NASH), a serious condition whose features include liver steatosis, liver inflammation, and hepatocyte ballooning. Left unaddressed, NASH may progress further to cirrhosis and or hepatocellular carcinoma, often resulting in liver transplant or death. NASH is a complex disease whose genesis is linked to a number of factors including genetics, metabolic syndrome, and/or external factors such as diet and exercise, making identification of new therapies challenging (Dongiovanni P, Anstee Q M, Valenti L. Curr Pharm Des. 2018, 19: 5219-38). As of today, there are no approved drug therapies for NASH, spurring a drug development race to fulfill an unmet need. The mechanisms of action of these NASH therapies under development are extremely varied and target very different aspects and stages of the disease process.

Various animal and cellular models have been proposed to elucidate pathological mechanisms of NAFLD and identify novel therapies. However, none of the models available satisfactorily mimic an in vivo-like liver model that would be physiologically relevant.

Therefore, there is still an unmet need of providing an in vitro model of NAFLD that mimics the physiological behavior of the liver occurring during NAFLD progression from steatosis to more severe disease states such as fibrosing NASH.

### SUMMARY OF THE INVENTION

The present invention relates to the provision of compositions and methods to solve these unmet needs.

Accordingly, the present invention relates to a first cell culture medium comprising:
at least one carbohydrate;
at least one free fatty acid;
insulin; and
a cholesterol source.

The invention also relates to a second cell culture medium comprising:
at least one carbohydrate;
at least one free fatty acid;
insulin;
a cholesterol source;
at least one growth factor superfamily member;
at least one tumor necrosis factor superfamily member; and
at least one sphingolipid family member.

In another aspect, the invention relates to a method for inducing a steatosis-like phenotype in a three-dimensional (3D) liver microtissue, comprising the step of culturing the 3D liver microtissue in the first cell culture medium of the invention. Another aspect of the invention relates to a 3D liver microtissue having a steatosis-like phenotype, obtainable according to this method.

In a further aspect, the invention relates to a method for inducing a fibrosing NASH-like phenotype in a 3D liver microtissue, comprising the step of culturing a 3D liver microtissue having a steatosis-like phenotype in the second cell culture medium of the invention. In a particular variant of this embodiment, a 3D liver microtissue is first (a) induced in a steatosis-like phenotype by culturing said 3D liver microtissue in the first cell culture medium of the invention; and then (b) the 3D liver microtissue induced according to step (a) is cultured in the second cell culture medium of the invention. A further aspect of the invention relates to a 3D liver microtissue having a NASH-like phenotype, obtainable according to this method.

The invention further relates to the use of a 3D liver microtissue having a steatosis-like phenotype as provided herein, for screening potential anti-steatosis substances.

The invention also relates to the use of a 3D liver microtissue having a fibrosing NASH-like phenotype as provided herein, for screening potential anti-NASH substances.

The invention also relates to a method for screening the potential anti-steatosis effect of a test substance, comprising:

i. inducing a steatosis-like phenotype in a 3D liver microtissue according to the method described herein, or providing a 3D liver microtissue having a steatosis-like phenotype according to the invention;

ii. contacting the 3D liver microtissue having a steatosis-like phenotype with said test substance; and

iii. determining the variation of at least one parameter in the 3D liver microtissue or in the culture medium following step ii.

The invention also relates to a method for screening the potential anti-fibrosing NASH effect of a test substance, comprising:

i. inducing a fibrosing NASH-like phenotype in a 3D liver microtissue according to the method described herein, or providing a 3D liver microtissue having a fibrosing NASH-like phenotype according to the invention;

ii. contacting the 3D liver microtissue having a fibrosing NASH-like phenotype with said test substance; and

iii. determining the variation of at least one parameter in the 3D liver microtissue or in the culture medium following step ii.

### LEGENDS OF THE FIGURES

FIG. 1 is a histogram showing steatose quantification with Adipored signal quantification in a 3D liver microtissue steatosis model of the invention.

FIG. 2 is a histogram showing the antisteatotic activity of MK-4074 in a 3D liver microtissue steatosis model of the invention.

FIG. 3 is a histogram showing the antisteatotic activity of CP-640186 in a 3D liver microtissue steatosis model of the invention.

FIG. 4 is a histogram showing the antisteatotic activity of GS0976 in a 3D liver microtissue steatosis model of the invention.

FIG. 5 is a histogram showing steatosis level after induction of a fibrosing NASH-like phenotype in a 3D liver microtissue according to the invention.

FIG. 6 is a histogram showing the level of MCP1 after induction of a fibrosing NASH-like phenotype in a 3D liver microtissue according to the invention.

FIG. 7 is a histogram showing the level of IL-8 after induction of a fibrosing NASH-like phenotype in a 3D liver microtissue according to the invention.

FIG. 8 is a histogram showing the level of pro-collagen I after induction of a fibrosing NASH-like phenotype in a 3D liver microtissue according to the invention.

FIG. 9 is a histogram showing the impact of drug substances on collagen release in a fibrosing NASH 3D liver microtissue according to the invention.

A. inhibition of COL1A1 secretion in medium 1+medium2-treated 3D liver microtissues treated with high doses of Elafibranor (ELA-3 μM), GS-0976 (1 μM) and CP-640186 (3 μM) At low doses, ELA synergizes with ACC inhibitors (B GS-0976, and C CP-640186) to attenuate COL1A1 secretion from medium1+medium2-treated 3D liver microtissues [ELA (0.3 μM), GS-0976 (0.3 μM), CP-640186 (0.3 μM)]. HSA: Highest than the best Single Agent.

### DETAILED DESCRIPTION OF THE INVENTION

### 3D Liver Microtissue Preparation

The present invention provides compositions and methods useful for the induction of 3D liver microtissues into relevant models of NAFLD.

A 3D liver microtissue is a three dimensional spheroid hepatic cell monoculture or coculture, having a physical structure close to a spherical structure with cells aggregated in suspension into the cell culture medium, with no contact of said culture with the culture support. A 3D liver microtissue elicits enhanced liver phenotype, metabolic activity, and stability in culture not attainable with conventional two-dimensional hepatic models.

The 3D liver microtissue used in the present invention comprises hepatocytes. These hepatocytes can be either monocultured, or cultured together with other cell types. Illustrative other cell types that can be co-cultured with hepatocytes include, without limitation, other hepatic cell types such as hepatic non-parenchymal cells, in particular hepatic stellate cells, Kupffer cells and liver endothelial cells. In a particular embodiment of the invention, the 3D liver microtissue is a co-culture of hepatocytes and hepatic non-parenchymal cells. In a further particular embodiment, the non-parenchymal cells included in the co-culture comprise hepatic stellate cells (HSCs), Kupffer cells and liver endothelial cells.

The hepatic cells comprised in the 3D liver microtissue can be primary hepatic cells, such as primary hepatocytes containing or not primary hepatic non-parenchymal cells. Primary hepatic cells can include cryopreserved primary hepatic cells or fresh primary hepatic cells. Alternatively, the hepatic cells can be hepatic cell lines, such as a hepatocyte-derived carcinoma cell lines, for example the Huh7 human hepatocellular carcinoma cell line, containing or not hepatic non-parenchymal cell lines.

The cells present in the 3D liver microtissue can be derived from mammalian cells, such as from human cells. In a particular embodiment, the cells comprised in the 3D liver microtissue are human cells. Accordingly, in a particular embodiment, the 3D liver microtissue comprises human hepatocytes, such as primary human hepatocytes or a human hepatocyte cell line such as the Huh7 cell line, and, in case of a co-culture, human hepatic non-parenchymal cells.

In a particular embodiment, the 3D liver microtissue is a monoculture of human hepatocytes, in particular of a human hepatocyte cell line such as the Huh7 cell line.

In another embodiment, the 3D liver microtissue is a co-culture of primary human hepatocytes and primary human hepatic non-parenchymal cells. In a further particular embodiment, the 3D liver microtissue is a co-culture of primary human hepatocytes, primary HSCs, primary Kupffer cells and primary liver endothelial cells.

3D liver microtissue can be prepared according to methods known in the art. For example, the methods described in Bell et al., 2016 (Bell et al., Sci Rep. 2016 May 4; 6:25187) can be used for the preparation of hepatocyte spheroid monocultures or co-cultures.

### Cell Culture Media of the Invention

The first cell culture medium of the invention comprises:
at least one carbohydrate;
at least one free fatty acid;
insulin; and
a cholesterol source.

This first cell culture medium can be useful for the induction of 3D liver microtissues into a steatosis-like phenotype.

The second cell culture medium of the invention comprises:
at least one carbohydrate;
at least one free fatty acid;
insulin;
a cholesterol source;
at least one growth factor superfamily member;
at least one tumor necrosis factor superfamily member; and
at least one sphingolipid family member.

The second cell culture medium can be particularly advantageous in inducing a 3D liver microtissue having a steatosis-like phenotype into a fibrosing-NASH-like phenotype.

The first cell culture medium and the second cell culture medium according to the invention can be derived from cell culture media well known in the art (also referred to herein as a “base culture medium”), supplemented with the above mentioned components. For example, the base culture medium can be a William's E cell culture medium or Dulbecco's Modified Eagle Medium (DMEM), or any other cell culture medium suitable for the culture of hepatocytes and hepatic non-parenchymal cells. The base culture medium can be supplemented with common components useful in cell culture, in particular liver cell culture, such as antibiotics, buffers and nutrients. Useful supplements include, without limitation, transferrin, selenium, glutamine, HEPES, gentamicin and amphotericin B. In a particular embodiment, the base culture medium is a Williams E cell culture medium supplemented with transferrin, selenium, glutamine, HEPES, gentamicin and amphotericin B.

The base culture medium used for the first and second cell culture medium of the present invention can be the same or different, in particular the same.

According to the invention, the term “carbohydrate” denotes sugars and polysaccharides. In a particular embodiment, a sugar may be selected from monosaccharides, disaccharides and polyols.

Illustrative monosaccharides useful in the practice of the present invention include glucose, galactose, fructose and xylose.

In addition, illustrative disaccharides useful in the practice of the present invention include sucrose, lactose, maltose and trehalose.

Illustrative polyols include, without limitation, sorbitol and mannitol.

Among the oligosaccharides that can be used in the context of the present invention, one can cite malto-oligosaccharides such as maltodextrins, or other oligosaccharides such as raffinose, stachyose and fructo-oligosaccharides.

Polysaccharides can also non-limitatively be selected from starch polysaccharides such as amylose, amylopectin and modified starches, and non-starch polysaccharides such as glycogen, cellulose, hemicellulose, pectins and hydrocolloids.

According to a particular embodiment of the invention, the at least one carbohydrate is selected from sugars, in particular from monosaccharides and disaccharides. In a further particular embodiment, the at least one carbohydrate is selected from monosaccharides.

In yet another embodiment, the at least one carbohydrate comprises at least two carbohydrates. In another embodiment, the at least two carbohydrates comprise glucose. According to an embodiment, the at least two carbohydrates comprise fructose. In another embodiment, the at least two carbohydrates comprise glucose and fructose.

The concentration of the at least one carbohydrate in the cell culture media can vary. For example, glucose can be at a concentration comprised between 1 and 50 mM in the cell culture medium, in particular between 2 and 25 mM, more particularly between 5 and 15 mM. In a particular embodiment, glucose concentration in the cell culture medium is of 10 mM. Fructose concentration may also vary, and can be comprised, in particular, between 1.5 and 100 mM, such as between 10 and 20 mM. In a particular embodiment, glucose concentration in the cell culture medium is of 15 mM. In a further particular embodiment, the first and the second culture media comprise glucose and fructose, wherein

glucose is at a concentration comprised between 1 and 50 mM in the cell culture medium, in particular between 2 and 25 mM, more particularly between 5 and 15 mM; and

fructose is at a concentration comprised between 1.5 and 100 mM, such as between 10 and 20 mM.

According to the invention, the term “free fatty acid” denotes saturated and unsaturated C10-C20 free fatty acids, conjugated to albumin.

Illustrative unsaturated C10-C20 free fatty acids include, without limitation, monounsaturated, diunsaturated, triunsaturated, tetraunsaturated and pentaunsaturated C10-C20 free fatty acids. In a particular embodiment, the unsaturated C10-C20 free fatty acids may be selected from trimyristoleic acid, palmitoleic acid, sapienic acid, oleic acid, elaidic acid, vaccenic acid, gadoleic acid, eicosenoic acid, dilinoleic acid, eicosadienoic acid, linolenic acid, gamma-linoleic acid, pinolenic acid, eleostearic acid, beta-eleostearic acid, mead acid, dihomo-gamma-linolenic acid, eicosatrienoic acid, stearidonic acid, arachidonic acid, eicosatetraenoic acid, bosseopentaenoic acid, and eicosapentaenoic acid.

Illustrative saturated C10-C20 free fatty acids include, without limitation, capric acid, undecanoic acid, lauric acid, tridecanoic acid, myristic acid, pentadecanoic acid, palmitic acid, margaric acid, stearic acid, nonadecanoic acid and arachidic acid.

In another embodiment, the at least one free fatty acid is selected from saturated or unsaturated C16-C20 free fatty acids.

In a particular embodiment, the at least one free fatty acid comprises at least one unsaturated free fatty acid such as oleic acid.

In another particular embodiment, the at least one free fatty acid comprises at least one saturated free fatty acid such as palmitic acid.

In a particular embodiment, the at least one free fatty acid comprises at least two free fatty acids. In a further embodiment, the at least two free fatty acids comprise oleic acid. In another embodiment, the at least two free fatty acids comprise palmitic acid. In yet another embodiment, the two free fatty acids comprises oleic acid and palmitic acid.

The concentration of the at least one free fatty acid can vary in the cell culture media of the invention. For example, each free fatty acid of the at least one free fatty acid in the cell culture medium can have a concentration comprised between 50 μM and 5 mM, such as between 100 μM and 1 mM. According to another embodiment, the at least one free fatty acid is a mix of free fatty acids, the total concentration of which (i.e. the sum of the concentration of each free fatty acid of the mix) is comprised between 50 μM and 5 mM, such as between 100 μM and 1 mM.

According to the invention, the term “cholesterol source” denotes soluble cholesterol or lipoprotein-cholesterol (i.e. high density lipoprotein (HDL)-cholesterol, low density lipoprotein (LDL)-cholesterol or very low density lipoprotein (VLDL)-cholesterol). In a particular embodiment, the cholesterol in the cell culture media of the invention is soluble cholesterol.

In a further embodiment, cholesterol is soluble cholesterol at a concentration comprised between 5 and 500 μg/mL in the culture medium.

In a further embodiment, lipoprotein-cholesterol is LDL-cholesterol at a concentration comprised between 5 and 500 μg/mL in the culture medium.

The concentration of insulin in the first or second culture medium can vary. In a particular embodiment, the concentration of insulin is comprised between 10 ng/mL and 10 μg/mL.

The second cell culture medium comprises at least one growth factor superfamily member. A growth factor is a naturally occurring substance capable of stimulating cellular growth, proliferation, healing, and cellular differentiation. Usually, it is a protein or a steroid hormone. Growth factors are important for regulating a variety of cellular processes. A non-limitative list of growth factors useful in the practice of the present invention includes a platelet-derived growth factor (PDGF), an epidermal growth factor (EGF) and a transforming growth factor (TGF). In a particular embodiment, the at least one growth factor superfamily member comprises a PDGF, such as PDGF-BB, PDGF-AB and PDGF-AA, more particularly PDGF-BB.

In a further particular embodiment, the at least one growth factor superfamily member is at a concentration comprised between 0.1 ng/mL and 10 ng/mL in the second cell culture medium.

The second cell culture medium also comprises at least one tumor necrosis factor superfamily member. The tumor necrosis factor (TNF) superfamily (TNFSF) is a protein superfamily of type II transmembrane proteins containing TNF homology domain and forming trimers. Members of this superfamily can be released from the cell membrane by extracellular proteolytic cleavage and function as cytokines. These proteins are expressed predominantly by immune cells and regulate diverse cell functions, including regulation of immune response and inflammation, but also proliferation, differentiation, apoptosis and embryogenesis. The superfamily contains 19 members that bind to 29 members of TNF receptor superfamily. In a particular embodiment, the TNF superfamily member is selected from TNFSF1, TNFSF2, TNFSF3, TNFSF4, TNFSF5, TNFSF6, TNFSF7, TNFSF8, TNFSF9, TNFSF10, TNFSF11, TNFSF12, TNFSF13, TNFSF13B, TNFSF14, TNFSF15 and TNFSF18. In a particular embodiment, the at least one TNFSF member comprises TNFSF14 (otherwise referred to as LIGHT protein).

In a further particular embodiment, the at least one TNFSF member is at a concentration comprised between 1 ng/mL and 100 ng/mL in the second cell culture medium.

The second cell culture medium also comprises a sphingolipids family member. In a particular embodiment, the sphingolipids family member is selected from ceramid, sphingomyelin, sphingosine, glycosphingolipids. In a particular embodiment, the sphingolipids family member is sphingosine-1-phosphate.

In a further particular embodiment, the sphingolipids family member is at a concentration comprised between 10 ng/mL and 1000 ng/mL in the second cell culture medium.

In certain embodiments, the first and second cell culture media comprise the same at least one carbohydrate, the same at least one free fatty acid and the same cholesterol source. For the sake of clarity, this means that if the first cell culture medium contains glucose as the at least one carbohydrate, oleic acid as the at least one free fatty acid, and soluble cholesterol as cholesterol source, then the second cell culture medium also comprises glucose as the at least one carbohydrate, oleic acid as the at least one free fatty acid, and soluble cholesterol as cholesterol source. Also for the sake of clarity, the fact that the first and second cell culture media comprise the same at least one carbohydrate, the same at least one free fatty acid and the same cholesterol source does not mean that they are at the same concentration in both media. In a particular embodiment, these components and insulin are at the same concentration in the first cell culture medium and in the second cell culture medium. However, in other embodiments, these components and the insulin are at different concentrations in the first cell culture medium and in the second cell culture medium. In particular, these common components between the first and second cell culture medium may be at a given concentration in the first culture medium, and twice concentrated (2×) in the second culture medium, as compared to the first culture medium. These common components can also be at a 3×, 4×, 5×, or even more than 5× concentration in the second cell culture medium, as compared to the concentration in the first culture medium. As provided below, this may be useful during the culturing steps.

Accordingly, in a particular embodiment of the invention, the present application specifically discloses all the specific concentrations and concentration ranges provided above and their 2-fold, 3-fold, 4-fold, 5-fold and even more than 5-fold values.

### Method for Inducing Steatosis-Like Phenotype

An aspect of the invention relates to the use of the first cell culture medium of the invention for inducing a steatosis-like phenotype in a 3D liver microtissue. The induction of a steatosis-like phenotype comprises a step of culturing a 3D liver microtissue in the first cell culture medium of the invention, for a time sufficient for inducing said steatosis-like phenotype. In a particular embodiment, the culture time is comprised between 24 hours and 240 hours, such as between 48 hours and 192 hours. In a further particular embodiment, the first cell culture medium may be replaced by fresh medium during the course of the induction, for example every 24 hours, every 48 hours or every 72 hours. In addition, medium change does not necessarily require complete medium change. For example, the medium change can comprise withdrawal of a third or half of the old culture medium, and addition of the same, fresh first culture medium comprising the at least one carbohydrate, at least one free fatty acid, insulin and cholesterol source as provided above.

In a further particular embodiment, the 3D liver tissue for use in the method for inducing a steatosis-like phenotype is a monoculture of hepatocytes, such as a monoculture of a hepatocyte cell line, in particular a monoculture of HuH7 cells.

The invention also relates to a 3D liver microtissue having a steatosis-like phenotype, obtainable according to this method. The 3D liver microtissue having a steatosis-like phenotype according to the invention can be used as a steatosis model.

### Method for Inducing a Fibrosing NASH-Like Phenotype

Another aspect of the invention relates to the use of the second cell culture medium of the invention for inducing a fibrosing NASH-like (f-NASH-like) phenotype in a 3D liver microtissue.

Various studies show that 20-50% of patients with NASH will display disease progression, either in the form of increased inflammation or liver fibrosis. Factors leading to progressive NASH and inflammation are not well understood, although it has been agreed upon that multiple ‘pathogenic hits’ are required for the progressive development of liver disease. Because liver fibrosis is characterized by the excessive deposition of extracellular matrix (ECM), markers which represent ECM components, like collagens, are widely employed to assess the development of liver fibrosis. The term ‘fibrosing NASH’ thus refers to NASH features plus collagen production.

The induction of a f-NASH-like phenotype comprises a step of culturing a 3D liver microtissue having a steatosis-like phenotype in the second cell culture medium of the invention, for a time sufficient for inducing said f-NASH-like phenotype. In a particular embodiment, the culture time in the second cell culture medium is comprised between 24 hours and 240 hours, such as between 48 hours and 192 hours. In a further particular embodiment, the second cell culture medium may be replaced by fresh medium during the course of the f-NASH-like phenotype induction, for example every 24 hours, every 48 hours or every 72 hours. In addition, medium change does not necessarily require complete medium change. For example, the medium change can comprise withdrawal of a third or half of the old second cell culture medium of the invention, and addition of the same, fresh second culture medium comprising the at least one carbohydrate, at least one free fatty acid, insulin and cholesterol as provided above.

In a particular embodiment of the invention, the method for inducing a f-NASH-like phenotype in a 3D liver microtissue comprises:

(a) culturing a 3D liver microtissue in the first cell culture medium of the invention; and then

(b) culturing the 3D liver microtissue in the second cell culture medium of the invention.

In a particular embodiment, step (a) is carried out for a duration between 24 and 120 hours, in particular between 48 and 96 hours, more particularly between 60 and 84 hours, such as between 70 and 74 hours.

In a further particular embodiment, step (b) is carried out for a duration between 24 and 120 hours, in particular between 48 and 96 hours, more particularly between 60 and 84 hours, such as between 70 and 74 hours.

In a further particular embodiment, both step (a) and step (b) are carried out for a duration between 70 and 74 hours.

In another embodiment, the first culture medium used in step (a) is replaced with the second culture medium for carrying out step (b) by total medium replacement. In this embodiment, the common components with the second cell culture medium comprise the at least one carbohydrate, at least one free fatty acid, insulin and cholesterol source as provided above at a 1× concentration, i.e. at the final concentration they should be when contacted with the 3D liver microtissue.

In an alternative embodiment, the first culture medium used in step (a) is replaced with the second culture medium for carrying out step (b) by partial medium replacement. As explained above, in this embodiment, the components of the second cell culture medium of the invention defined above (i.e. the at least one carbohydrate, at least one free fatty acid, the insulin, the cholesterol source, the growth factor superfamily member, the TNFSF member and the sphingolipid family member) are at a higher concentration, as compared to their desired final concentration for culturing the 3D liver microtissue. For example, the medium change can comprise withdrawal of a third or half of the old first cell culture medium of the invention from the culture, and addition of fresh second culture medium comprising a three-fold concentration or two-fold concentration, respectively, of the at least one carbohydrate, at least one free fatty acid, the insulin, the cholesterol, the growth factor superfamily member, the TNFSF member and the sphingolipid family member as compared to the desired final concentration of these components for culturing the 3D liver microtissue.

The invention also relates to a 3D liver microtissue having a f-NASH-like phenotype, obtainable according to this method. The 3D liver microtissue having a f-NASH-like phenotype according to the invention can be used as a f-NASH model.

### Method for Screening Therapeutic Substances

In another aspect, the invention relates to a method for screening the potential anti-steatosis effect of a test substance, comprising:

culturing the 3D liver microtissue having a steatosis-like phenotype according to the invention with said test substance; and

determining the variation of at least one parameter in the 3D liver microtissue or in the culture medium.

More particularly, the method for screening the potential anti-steatosis effect of a test substance, can comprise:

i. inducing a steatosis-like phenotype in a 3D liver microtissue according to the method described herein, or providing a 3D liver microtissue having a steatosis-like phenotype according to the invention;

ii. culturing the 3D liver microtissue having a steatosis-like phenotype in the presence of said test substance; and

iii. determining the variation of at least one parameter in the 3D liver microtissue or in the culture medium.

However, it should be understood that steps i. and ii. can also be carried out at the same time, or step ii. can be implemented before step i., i.e. contacting the 3D liver microtissue with the test substance does not necessarily occur after induction of the steatosis-like phenotype.

The invention also relates to a method for screening the potential anti-f-NASH effect of a test substance, comprising:

culturing the 3D liver microtissue having a f-NASH-like phenotype according to the invention with said test substance; and

determining the variation of at least one parameter in the 3D liver microtissue or in the culture medium.

In a particular embodiment, the method for screening the potential anti-f-NASH effect of a test substance, comprises:

i. inducing a f-NASH-like phenotype in a 3D liver microtissue according to the method described herein, or providing a 3D liver microtissue having a f-NASH-like phenotype according to the invention;

ii. contacting the 3D liver microtissue having a f-NASH-like phenotype with said test substance; and

iii. determining the variation of at least one parameter in the 3D liver microtissue or in the culture medium following step ii.

However, it should be understood that steps i. and ii. can also be carried out at the same time, or step ii. can be implemented before step i., i.e. contacting the 3D liver microtissue with the test substance does not necessarily occur after induction of the f-NASH-like phenotype.

The test substance can be of any type, such as a small molecule, a macromolecule or more complex substances such as viruses or parasites. Macromolecules include, without limitation, peptides, proteins and nucleic acids.

The test substance can be part of a library of substances, for example a library of small molecules or a library of macromolecules.

The method of the invention can also be used to assess the effect of combinations of substances.

The effect of the test substance can be assessed by determining the variation of at least one parameter in the 3D liver microtissue or in the culture medium of the 3D liver microtissue culture. The at least one parameter can be selected from markers relevant to the particular disease the model is used for. In particular, the at least one parameter can be selected from steatosis, fibrosis and inflammation markers.

Illustrative steatosis markers that may be assessed include, without limitation: increased lipogenesis, desaturases, LOX activities, impaired peroxisomal polyunsaturated fatty acid (PUFA) metabolism.

Illustrative fibrosis markers that may be assessed include, without limitation: collagen, elastin, glycoproteins, hyaluronan, Tissue Inhibitors of MetalloProteinases (TIMPs), Cartilage oligomeric matrix protein (COMP).

Illustrative inflammation markers that may be assessed include, without limitation: Interleukin-8 (IL-8), monocytes chemoattractant protein-1 (MCP-1), tumor necrosis factor-α (TNF-α), interleukin-6 (IL-6), Interleukin 1 beta (IL-1β), Chemokine (C—C motif) ligand 5 (CCL-5).

In particular, the at least one parameter may be selected from the group consisting of protein markers, RNA markers, 3D liver microtissue size and 3D liver microtissue integrity. Other parameters include lipid content of the 3D liver microtissue, chemokines production, cytokines production and collagen production.

### Kits of the Invention

Another aspect of the invention relates to a kit comprising the first cell culture medium and/or the second culture medium of the invention. In a particular embodiment, the kit comprises both the first culture medium and the second culture medium of the invention.

The kit of the invention is useful for the implementation of a method of the invention. Accordingly, it may further include instructions to follow to carry out the invention.

In addition, the kit can further include other elements useful in the practice of the invention. These elements can include, without limitation:
one or several cell culture supports useful into which the 3D liver microtissue can be cultured, such as cell culture plates, for example 6-well, 12-well, 24-well, 48-well, 96-well, 192-well or 384-well culture plates; and/or
one or several buffers useful in the practice of the invention.

### EXAMPLES

### Example 1

### Steatosis Model

### Material and Methods

Cell Culture

The Huh 7 human hepatocellular carcinoma cell line was seeded in William's E medium (Gibco) with 10% FBS (Gibco) at a density of 3000 cells/well in 384 well plate ULA (Corning). After a few days, spheroids are formed and cells are grown in serum-free culture medium.

Metabolic Induction of Steatosis in 3D Huh7 Spheroids

William's E medium was supplemented with FFAs (oleate+palmitate, 400 μM total FFA concentration) fructose 15 mM (Sigma) and soluble cholesterol 50 m μg/mL (Sigma). Huh7 spheroids were treated for 3 days with this medium with or without a test compound and the treatment was renewed once with or without a test compound.

Steatosis was then measured after 6 days of treatment with or without the test compound.

The test compounds were acetyl Co-A carboxylase inhibitors.

Steatosis Measurement

Lipid Staining and Quantification Intracellular lipid accumulation was quantified using the AdipoRed™ Adipogenesis Assay Reagent (Lonza). Cells were visualized under epifluorescence microscope and then subjected to fluorescence assay quantification at λexc: 485 nm and λem: 572 nm, using plate reader (TECAN).

### Results

Steatosis Induction and Quantification

The results are reported in FIG. 1. It shows that the 3D liver microtissue cultured into a cell culture medium corresponding to the first culture medium according to the invention contains a significantly higher content of lipids as compared to untreated 3D liver microtissue. This experiment thus demonstrates that a steatosis-like phenotype may be induced thanks to the invention.

Inhibition of Steatosis with ACCi

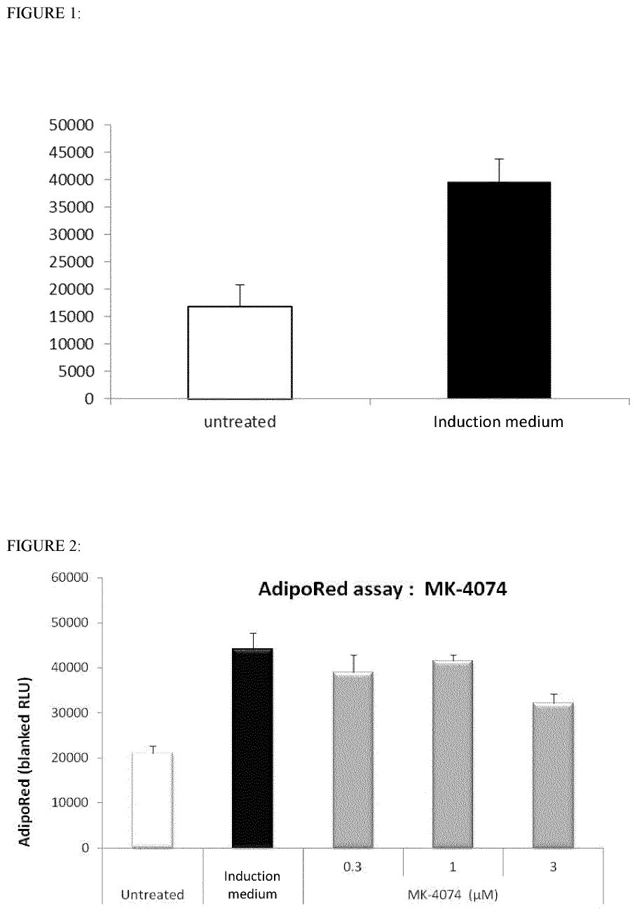

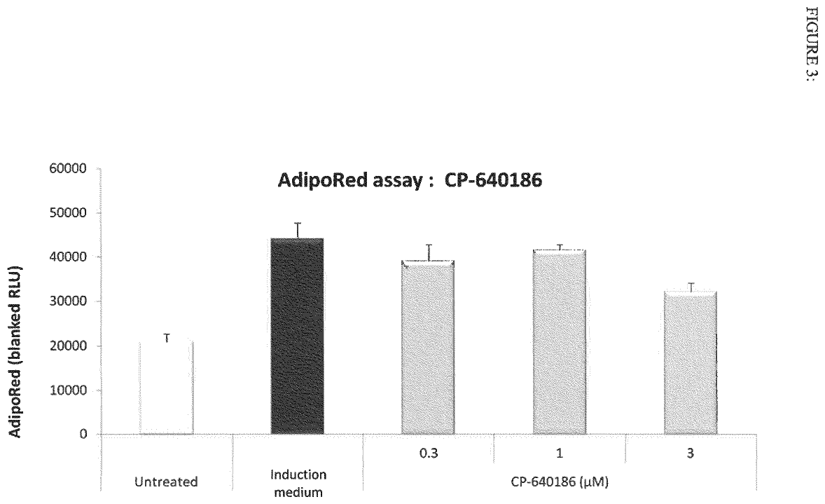

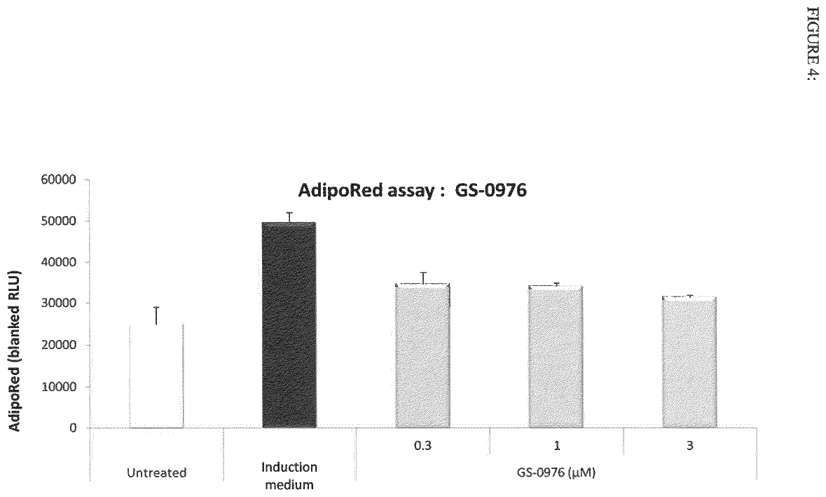

FIGS. 2 to 4 present the lipid content of 3D liver microtissue induced into a steatosis phenotype as provided above, further cultured in the present of different AAC inhibitors, i.e. MK-4074, CP-640186 and GS-0976, respectively.

The data reported in these figures show that ACC inhibitors under clinical development efficiently decreased the steatosis-like phenotype of the 3D liver microtissue model of steatosis according to the invention. The relevance of the model is thus validated.

### Example 2

### Fibrosing NASH Model

### Material and Methods

3D Human Liver Microtissue Culture

Cryopreserved primary human hepatocytes (IPHH_11) and cryopreserved primary human non parenchymal cells (NPCs containing endothelial and Kupffer cells) (IPHN_11) were obtained from BioIVT. The cryopreserved human primary hepatic stellate cells (hHSC) were obtained from Innoprot. 3D human liver microtissues were produced with the IPHH_11, the IPHN_11 and the hHSC in a 96-well hanging-drop culture platform (Gravity PLUS™, InSphero AG). After microtissues formation, they were transferred into a microtissue-specific 96-well culture and assay platform (Gravity TRAP™, InSphero AG). Further maintenance and compound treatments were performed in Gravity TRAP™ plates. After tissue formation, the 3D microtissues were maintained in William's E medium (Gibco) supplemented with Insulin (Sigma), Transferrin (Sigma), Selenium (Sigma), Glutamine (Gibco), Dexamethasone (Sigma), HEPES (Sigma), Gentamicin (Gibco) and Amphotericin B (Sigma) at 37° C. in a humidified 5% CO₂ cell culture incubator for 4 days. Half of the culture medium was replenished every 3 days.

Metabolic Induction of NASH in 3D Human Liver Microtissue

Hepatic microtissues were treated once with a couple of carbohydrates (final concentration ranging from 1 to 100 mM), a couple of free fatty acids either saturated or unsaturated with a final concentration ranging from 100μM to 1 mM and a source of cholesterol with a concentration ranging from 1 μg/mL to 1 mg/mL (medium 1). After 2 to 5 days, microtissues were treated with a couple of carbohydrates (final concentration ranging from 1 to 100 mM), a couple of free fatty acids either saturated or unsaturated with a final concentration ranging from 100 μM to 1 mM and a source of cholesterol with a concentration ranging from 1 μg/mL to 250 μg/mL, a member of the growth factor superfamily member, a member of the TNFSF with a concentration ranging from 1 ng/mL to 1 μg/mL and a sphingolipid with a concentration ranging from 1 nM to 500 nM (medium 2). This treatment lasts from 2 to 5 days.

Steatosis Measurements

Spheroids are stained with AdipoRed (Lonza) after 6 days of treatment with the metabolic induction of NASH. Spheroids are fixed (10% formalin) and then observed by epifluorescence microscopy (Olympus) to quantify fluorescence on images with ImageJ software.

ELISA Assays

The level of Collal, IL-8 and MCP-1 was measured using a Sandwich ELISA, according to manufacturer's instructions provided with “Elisa Pro-Collagen I α1/COLIA1”, (DuoSet ELISA, R&D, catalog N°: DY6220-05), Elisa CXCL8/IL-8 (DuoSet ELISA, R&D, catalog N°: DY208n) and Elisa CCL2/MCP-1 (DuoSet ELISA, R&D, catalog N°: DY279) kits, respectively.

### Results

NASH Features Induced After 6 Days of Metabolic Induction Phenotype:

### Steatosis

3D human liver microtissues are stained with AdipoRed after 6 days of treatment with medium 1+medium 2. 3D human liver microtissues are then observed by fluorescence microscopy to quantify fluorescence on images with ImageJ software.

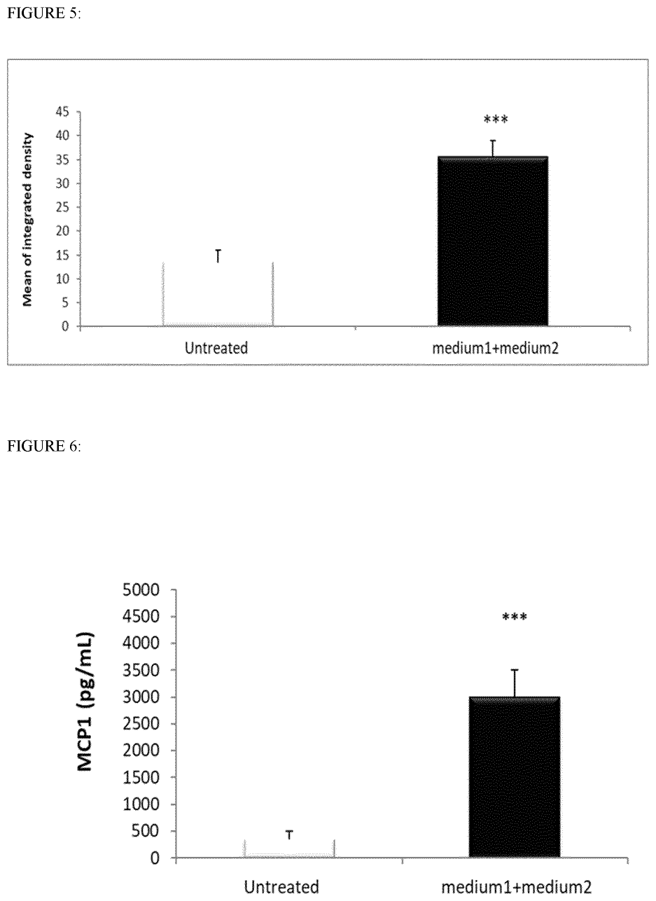

The data reported in FIG. 5 show that steatosis has been induced thanks to the method of the invention.

### Inflammation

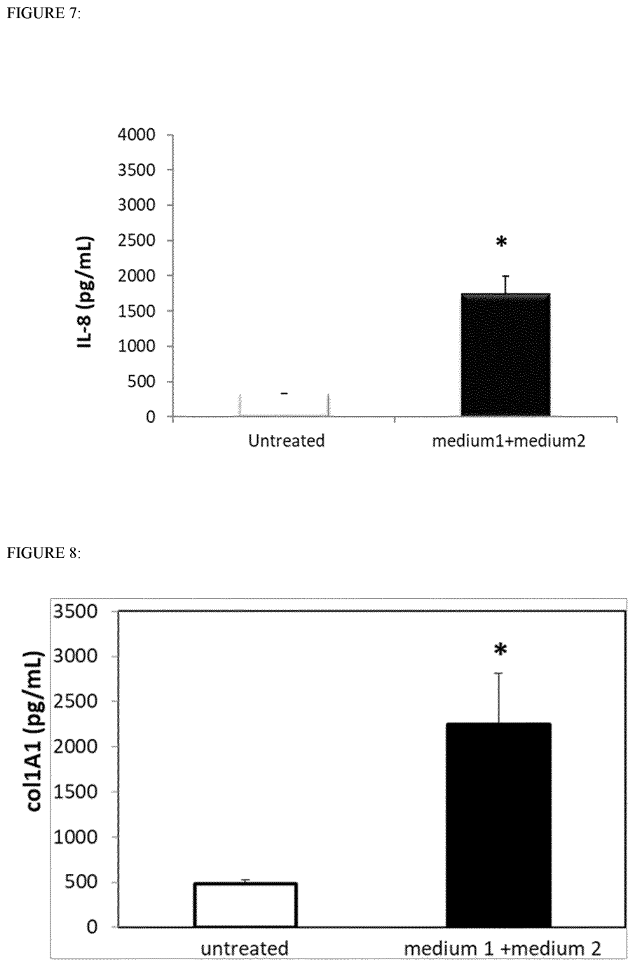

The data reported in FIGS. 6 and 7 show that inflammation has been induced thanks to the method of the invention.

### Fibrosis

Pro-Collagen I (Col1α1is a marker of fibrosis. FIG. 8 shows that the method of the invention induces fibrosis in the 3D liver microtissue, thereby reproducing a key feature of fibrosing NASH.

### Test Compound Evaluation:

2 Components Combination Matrix

For these experiments, a checkerboard matrix was generated. ELA and component (ii) stocks were serially diluted in DMSO in 2-points series in a row (ELA) and a 3-points series in a column (component (ii)) of a 96-well plate. Subsequently, the 2×3 combination matrix was generated by 1:1 mixing of all single agent concentrations.

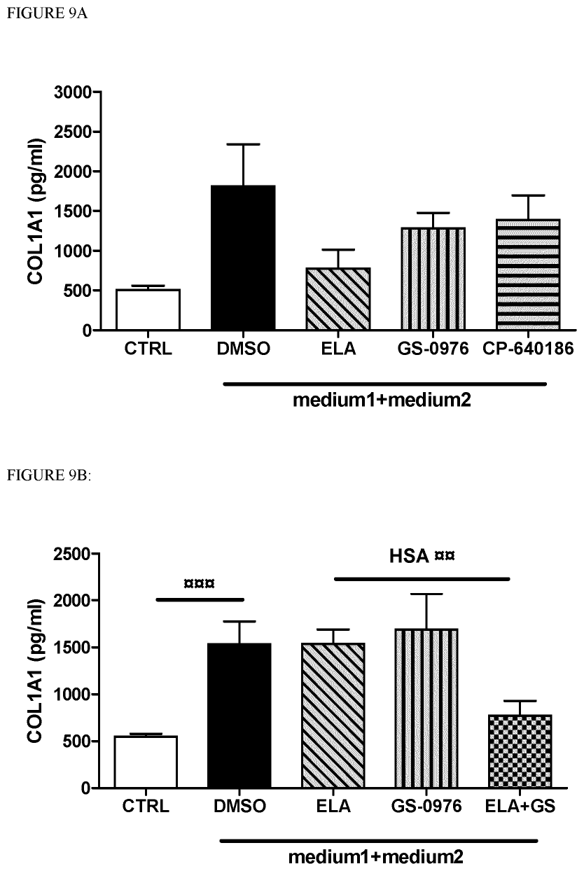

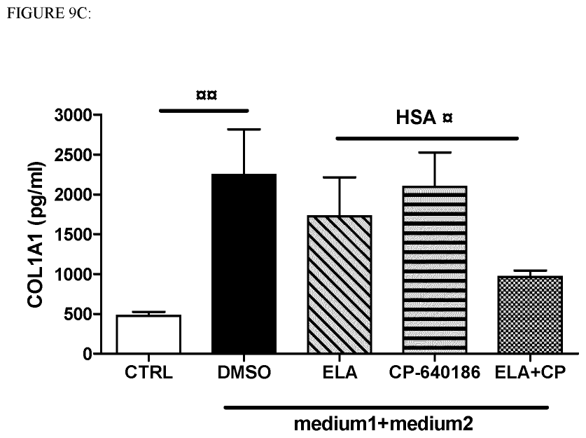

At high drug concentrations, the release of collagen was affected by either ELA or ACC inhibitors (GS-0976 and CP-640186) if applied as single treatments (FIG. 9A), confirming the physiological relevance of our model for the screening of anti-fibrosing NASH drugs. We also tested drugs belonging to the pipeline of anti-NASH molecules under development at low drug concentration to be able to observe a synergistic effect with drug combinations. Single drugs used at low dose did not elicit an effect on collagen release, but a potent inhibition of collagen release was observed if ELA and ACCi were used in combination on our 3D liver microtissue model used as a model of fibrosing NASH.

1-17. (canceled)

18. A cell culture medium comprising:
at least one carbohydrate;
at least one free fatty acid;
insulin;
a source of cholesterol;
at least one growth factor superfamily member;
at least one tumor necrosis factor superfamily member; and
at least one sphingolipid family member.

19. The cell culture medium according to claim 18, wherein the at least one carbohydrate is selected from the group consisting of sugars and polysaccharides.

20. The cell culture medium according to claim 18, wherein said at least one carbohydrate is at a concentration between 1 and 100 mM in said cell culture medium.

21. The cell culture medium according to claim 18, wherein the at least one free fatty acid is selected from the group consisting of C10-C20 unsaturated free fatty acids and C10-C20 unsaturated free fatty acids.

22. The cell culture medium according to claim 18, wherein the at least one free fatty acid is a mix of free fatty acids with a concentration between 50 μM and 5 mM in said cell culture medium.

23. The cell culture medium according to claim 18, wherein insulin is at a concentration between 10 ng/mL and 10 μg/mL in said cell culture medium.

24. The cell culture medium according to claim 18, wherein the source of cholesterol is soluble cholesterol at a concentration between 5 and 500 μg/mL in said cell culture.

25. The cell culture medium according to claim 18, wherein the growth factor superfamily member is at a concentration between 0.1 ng/mL and 10 ng/mL in the cell culture medium.

26. The cell culture medium according to claim 18, wherein the tumor necrosis factor superfamily member is at a concentration between 1 ng/mL and 100 ng/mL in the cell culture medium.

27. The cell culture medium according to claim 18, wherein the sphingolipid family member is sphingosine-1-phosphate at a concentration between 10 ng/mL and 1000 ng/mL in the cell culture medium.

28. A method for inducing a steatosis-like phenotype in a three-dimensional (3D) liver microtissue, comprising the step of culturing the 3D liver microtissue in a cell culture medium comprising at least one carbohydrate, at least one free fatty acid, insulin and a source of cholesterol.

29. A method for inducing a fibrosing non-alcoholic steatohepatitis (f-NASH)-like phenotype in a three-dimensional (3D) liver microtissue, comprising the following steps:
(a) culturing the 3D liver microtissue in a cell culture medium comprising at least one carbohydrate, at least one free fatty acid, insulin and a source of cholesterol; and then
(b) culturing the 3D liver microtissue in the cell culture medium as defined in claim 18.

30. A 3D liver microtissue having a steatosis-like phenotype, obtainable with the method according to claim 28.

31. A 3D liver microtissue having a fibrosing NASH-like phenotype, obtainable with the method according to claim 29.

32. A method for screening the potential anti-steatosis effect of a test substance, comprising:
i) inducing a steatosis-like phenotype in a 3D liver microtissue according to the method of claim 28;
ii) contacting the 3D liver microtissue having a steatosis-like phenotype with said test substance; and
iii) determining the variation of at least one parameter in the 3D liver microtissue or in the culture medium following step ii.

33. A method for screening the potential anti-fibrosing NASH effect of a test substance, comprising:
i) inducing a fibrosing NASH-like phenotype in a 3D liver microtissue according to the method of claim 29;
ii) contacting the 3D liver microtissue having a fibrosing NASH-like phenotype with said test substance; and
iii) determining the variation of at least one parameter in the 3D liver microtissue or in the culture medium following step ii.

34. A kit comprising:
a cell culture medium comprising at least one carbohydrate; at least one free fatty acid; insulin; and a source of cholesterol; and
the cell culture medium according to claim 18.

35. A method for screening the potential anti-steatosis effect of a test substance, comprising:
i) providing a 3D liver microtissue having a steatosis-like phenotype according to claim 30;
ii) contacting the 3D liver microtissue having a steatosis-like phenotype with said test substance; and
iii) determining the variation of at least one parameter in the 3D liver microtissue or in the culture medium following step ii.

36. A method for screening the potential anti-fibrosing NASH effect of a test substance, comprising:
i) providing a 3D liver microtissue having a fibrosing NASH-like phenotype according to claim 31;
ii) contacting the 3D liver microtissue having a fibrosing NASH-like phenotype with said test substance; and
iii) determining the variation of at least one parameter in the 3D liver microtissue or in the culture medium following step ii.
